(19) United States
(12) Patent Application Publication
(10) Pub. No.: US 2007/0212419 A1
(43) Pub. Date: Sep. 13, 2007

(54) Synthesis of biocompatible nanocomposite hydrogels as a local drug delivery system

(76) Inventors: Jozsef Bako, Nyinnegzes (HU); Marta Szepesi, Debrecen (HU); Adrienn J. Veres, Nagyecsed (HU); Csaba Cserhati, Debrecen (HU); Zsuzsa Maria Borbely, Debrecen (HU); Csaba Hegdus, Debrecen (HU); Janos Borbely, Debrecen (HU)

Correspondence Address: Thomas A. O'Rourke;Bodner & O'Rourke, LLP, 425 Broadhollow Road, Melville, NY, 11747, US

(21) Appl. No.: 11/707,653

(22) Filed: Feb. 17, 2007

Related U.S. Application Data
(60) Provisional application No. 60/774,470, filed on Feb. 18, 2006.

(51) Int. Cl. A61K 9/14; A61K 31/205

(52) U.S. Cl. 424/487; 977/906; 514/554

(57) ABSTRACT

Nanocomposite biocompatible hydrogels (NCHGs) may be synthesised as model systems for in situ cured local drug delivery devices for treatment of inter alia periodontal infections. The composite includes the following components: nanoparticles (NPs), a matrix gel, and chlorhexidine (CHX) or other antibacterial drug. The NPs were obtained by free radical initiated copolymerization of the monomers, 2-hydroxyethyl methacrylate (HEMA) and polyethyleneglycol dimethacrylate (PEGDMA), in aqueous solution. The same monomers were used to prepare crosslinked matrices by photopolymerization. NCHGs were obtained by mixing NPs, monomers, and drug in an aqueous solution then crosslinked by photopolymerization.

This application claims priority on U.S. Application Ser. No. 60/774,470 filed Feb. 18, 2006, the disclosures of which are incorporated herein by reference.

### FIELD OF THE INVENTION

The present invention is directed to the field of nanoparticle containing hydrogel compositions that may be used in drug delivery systems.

### BACKGROUND OF THE INVENTION

Hydrogels are three dimensional hydrophilic crosslinked polymer networks, which capable of swelling in water. They have soft and elastic nature, which suggests similarities to natural tissues. M Zrinyi, Magnetic field sensitive polymer gels, Trends in Polym. Sci. 5(9) (1997) 280-285. Their widespread application is well known, for example in the cosmetic industry as moisturising creams and emollients. Hydrogels have important role in ophthalmology as contact lenses, J. Gispet, R. Sola, C. Varon, The influence of water content of hydrogel contact lenses when fitting patients with ‘tear film deficiency’, Cont Lens Anterior Eye. 23(1) (2000) 16-21, or as a local drug delivery system to treat glaucoma, G-H. Hsiue, J-A. Guu, C-C. Cheng, Poly(2-hydroxyethyl methacrylate) film as a drug delivery system for pilocarpine, Biomaterials. 22 (2001) 1763-1769. In dermatology hydrogels are for rehydrating necrotical crusts. V Jankunas, R. Rimdeika, L. Pilipaityte, Treatment of the leg ulcers by skin grafting. Medicina (Kaunas). 40(5) (2004) 429-433. Hydrogels are also important in dentistry. For example, PerioChip is a hydrogel, used in periodontology as a drug delivery device for the release of CHX. P. A. Heasman, L. Heasman, F. Stacey, G. I. McCracken, Local delivery of chlorhexidine gluconate (PerioChip) in periodontal maintenance patients, J Clin Periodontol. 28(1) (2001) 90-95. Because of its efficiency, allowing reduction in drug dosage among other advantages, the application of controlled release systems is growing. Commonly, these involve matrices composed of biocompatible polymers. K. E. Uhrich, S. M. Cannizzaro, R. S. Langer, K. M. Shakesheff, Polymeric Systems for Controlled drug release, Chem Rev.c 99 (1999) 3181-3198; N. A. Peppas, P. Bures, W. Leobandung, H. Ichikawa, Hydrogels in pharmaceutical formulations, Eur J Pharm Biopharm. 50 (2000) 27-46; A. Das, S. Wadhwas, A. K. Srivastava, Cross-linked guar gum hydrogel discs for colon-specific delivery of Ibuprofen: formulation and in vitro evaluation, Drug Deliv. 13(2) (2006) 139-142; D-Y. Lee, L. S. W. Spangberg, Y-B. Bok, C-Y. Lee, K-Y. Kum, The sustaining effect of three polymers on the release of clorhexidine from a controlled release drug device for root canal disinfection, Oral Surg Oral Med. 100 (2005) 105-111; A. Jain, Y-T. Kim, R. J. McKeon, R. V. Bellamkonda, In situ gelling hydrogels for conformal repair of spinal cord defects, and local delivery of BDNF after spinal cord injury. Biomaterials. 27 (2006) 497-504; P. D. Riggs, M. Braden, M. Patel, Chlorhexidine release from room temperature polymerising methacrylate systems. Biomaterials 21 (2000) 345-351.

Mono and multifunctional acrylates and their derivatives are commonly used monomers for synthesis of biocompatible hydrogels. The synthesis methods can vary, for instance thermal—C. W. Huang, Y. M. Sun, W. F. Huang, Curing kinetics of the synthesis of poly (2-hydroxiethyl methacrylate) (PHEMA) with ethylene glycol dimethacrylate (EGDMA) as a crosslinking agent, J Polym Sci, Part A: Polym Chem. 35 (1997) 1873-1889, oxidation-reduction-K. Podual, F. J. Doyle III, N. A. Peppas, Dynamic behavior of glucose oxidase-containing microparticles of poly(ethylene glycol)-grafted cationic hydrogels in an environment of changing pH, Biomaterials. 21 (2000) 1439-1450, or photopolymerization—T. W. Atkins, R. L. McCallion, B. J. Tighe, The incorporation and sustained release of bioactive insulin from a bead-formed macroporous hydrogel matrix, J Biomed Mater Res. 29 (1995) 291-298; K. S. Anseth, R. A. Scott, N. A. Peppas, Effects of Ionization on the Reaction Behavior and Kinetics of Acrylic Acid Polymerizations, Macromolecules. 29 (1996) 8308-8312. The use of the photopolymerization is growing, and is replacing older methods. M. Friedman, G. Golomb, New sustained release dosage form of chlorhexidine for dental use. I Development and kinetics of release, J Periodontal Res. 17(3) (1982) 323-328; D. Steinberg, M. Friedman, A. Soskolne, M. N. Sela, A new degradable controlled release device for treatment of periodontal disease: in vitro release, J Periodontol. 61(7) (1990) 393-398; S. Lu, K. S. Anseth, Photopolymerization of mutilaminated poly(HEMA) hydrogels for controlled release, J Control Release. 57 (1999) 291-300; L. C. Yue, J. Poff, M. E. Cortes, R. D. Sinisterra, C. B. Faris, P. Hildgen, R. Langer, V. P. Shastri, A novel polymeric chlorhexidine delivery device for the treatment of periodontal disease, Biomaterials. 25 (2004) 3743-3750; D. Leung, D. A. Spratt, J. Pratten, K. Gulabivala, N. J. Mordan, A. M. Young, Chlorhexidine—release methacrylate dental composite materials, Biomaterials. 26 (2005) 7145-7153; J. Bako, M. Szepesi, A. J. Veres, Z. M. Borbely, C. Hegedus, J. Borbely, Chlorhexidine release from nanocomposit hydrogels, Polym Mat: Sci & Eng. 94 (2006) 367-368.

Among the large number of monomers that are available, HEMA is well known and is commonly used as a crosslinker, providing a very good biocompatible system. The properties of gels based on this monomer were modified by applying other materials for example different crosslinkers, and recently the use of nanoparticles is exponentially increasing. K. S. Soppimath, T. M. Aminabhavi, A. R. Kulkarni, W. E. Rudzinski, Biodegradable polymeric nanoparticles as drug delivery device, J Control Release. 70 (2001) 1-20; S. A. Agnihotri, N. N. Mallikarjuna, T. M. Aminabhavi, Recent advances on chitosan-based nanoparticles in drug delivery, J Control Release. 100 (2004) 5-28; X. Xia, Z. Hu, M. Marquez, Phisically bonded nanoparticle networks: a novel drug delivery system, J Control Release. 103 (2005) 21-30; Y-I. Chung, G. Tae, S. H. Yuk, A facile method to prepare heparin-functionalized nanoparticles for controlled release of growth factors, Biomaterials. 27 (2006) 2621-2626; D-H. Kim, D. C. Martin, Sustained release of dexamethasone from hydrophilic matrices using PLGA nanoparticles for neutral drug delivery, Biomaterials. 27 (2006) 3031-3037; O. M. Koo, I. Rubinstein, H. Onyuksel, Role of nanotechnology in targeted delivery and imaging: a concise review, Nanomedicine. 1 (2005) 193-212. The opportunity of utilization nanocomposites is an intensively researched area in the bone grafting, or tissue regeneration, R. Murugan, S. Ramakrishna, Development of nanocomposites for bone grafting, Compos Sci Technol. 65 (2005) 2385-2406; A. Sinha, G. Das, B. K. Sharma, R. P. Roy, A. K. Pramanick, S. Nayar, Poly(vinyl alcohol)-Hydroxyapatite biomimetic scaffold for tissue regeneration, Mat Sci Eng C. xx (2006) xxx-xxx (available on line), and the novel hydrogel nanocomposites are also investigated. Z. Weian, L. Wei, F. Yue'e, Synthesis and properties of a novel nanocomposites, Mat Lett. 59 (2005) 2876-2880; M. Z. Hussein, A. H. Yahaya, Z. Zainal, L. H. Kian, Nanocomposite-based controlled release formulation of an herbicide, 2,4-dichlorophenoxyacetate incapsulated in zinc-aluminium-layered double hydroxide, Sci Techn Adv Mat. 6 (2005) 956-962.

### SUMMARY OF THE INVENTION

Nanocomposite hydrogels may be prepared by incorporation of nanoparticles into the gel matrix. The integrated gel system showed distinct advantages compared to simple hydrogels as drug delivery systems. The swelling ratio of NCHG is up to 200% related to the solid content and results in a flexible, soft gel for implantation into the periodontal pockets or for application as a surface film on infected gums. The compression strength increased with higher content of the cross linker, PEGDMA. Adding NPs to the matrix this value remains constant. The release slope of CHX declined for NCHG, indicating a slower release of the drug from the composite hydrogels. When CHX was excluded from NPs incorporated in the gel, an unexpected apparent decline in the released drug was observed. In situ polymerization of hydrogels offers flexibility for local placement of drugs in the treatment of, for example, periodontal disease.

Nanocomposite biocompatible hydrogels (NCHGs) were synthesised as model systems for in situ cured local drug delivery devices for treatment of periodontal infections. The composite includes the following components: nanoparticles (NPs), a matrix gel, and chlorhexidine (CHX) as antibacterial drug. The NPs were obtained by free radical initiated copolymerization of the monomers, 2-hydroxyethyl methacrylate (HEMA) and polyethyleneglycol dimethacrylate (PEGDMA), in aqueous solution. The same monomers were used to prepare crosslinked matrices by photopolymerization. NCHGs were obtained by mixing NPs, monomers, and drug in an aqueous solution then crosslinked by photopolymerization. It was found that compression strength values increased with increasing ratio of the crosslinker PEGDMA. Incorporation of NPs into the matrix resulted similar compression strength as the matrix hydrogel. The hydrated NCHGs swelled more slowly but admitted more water. Studies of release kinetics revealed that on average 60% of the loaded drug was released. The most rapid release was observed over a 24 hour period for matrix gels with low crosslinking density. For NCHGs the release period exceeded 48 hours. An unexpected result was observed for NCHGs without drug in the NPs. In this case, increasing release was observed for the first 24 hours. Thereafter, however, the apparent quantity of detectable drug decreased dramatically.

### BRIEF DESCRIPTION OF THE DRAWINGS

FIG. 1 shows the following SEM micrographs. a.) NPs of 5/5 HEMA/PEGDMA. b.): Matrix gel surface with a composition of HEMA/PEGDMA=5/5 (mold side surface), and c.): NCHG with a composition of 66% HEMA/PEGDMA=5/5 and 33% NPs (top side). d.) Broken NCHG of 66% HEMA/PEGDMA=5/5 and 33% NPs.

FIG. 2 demonstrates the effect of the ratio of crosslinker in the matrix and that of the NPs for the compression properties of the hydrogels. Black arrow indicates two samples (M50-50 matrix and NCHG nanocomposite hydrogel) with the same composition of matrix gel (HEMA/PEGDMA=5/5) however, the NCHG sample consists of 30% of NPs.)

FIG. 3 shows the swelling properties of the 50% HEMA 50% PEGDMA hydrogel (matrix), and the NCHG composed of 66% 50/50 HEMA/PEGDMA hydrogel and 33% of 50/50 HEMA/PEGDMA NPs.

FIG. 4 shows release profiles of matrix gel (50/50 HEMA/PEGDMA), and the NCHG (66% of 50/50 HEMA/PEGDMA hydrogel and 33% of 50/50 HEMA/PEGDMA NPs).

### DETAILED DESCRIPTION OF THE INVENTION

Materials and Methods

Materials

2-Hydroxyethyl methacrylate (97%, from Sigma-Aldrich, Steinheim, Germany) was purchased as monomer, poly(ethylene glycol) dimethacrylate (Mn:550, from Sigma-Aldrich, St. Louis, MO) as crosslinker and anthraquinone-2-sulfonic sodium salt (˜99%, Fluka AG. Buchs SG) as photoinitiator was applied. Chlorhexidine-di-gluconate, dental application grade (20% solution from Spektrum 3D, Hungary), was obtained as active substance. The composed of nanoparticles were done same monomers as the hydrogels, but the initiator was potassium persulphate (Reanal Co., Budapest, Hungary, 98% purity). Sodium-lauryl sulphate was applied as an emulsifier, and n-buthanole as cotenside were purchased from Spektrum 3D, Hungary. All materials were used as received without further purification.

Preparation of Nanoparticles

HEMA and PEGDMA monomers were dispersed in deionised water (40 ml) containing sodium lauryl sulphate (SLS), an anionic surfactant. The overall concentration of monomers was 4 wt %, and the concentration of SLS was 2.4 wt %. The composition feed of monomers was 50 mol % of HEMA and 50 mol % of PEGDMA. The batch copolymerization was performed in a three-necked, round-bottom flask. It was purged with nitrogen for 25 minutes on ambient temperature to remove the oxygen dissolved. A solution of potassium persulphate initiator (the concentration was 0.2 wt %) was added and free radical polymerization was performed at 60° C. The reaction mixture was continuously stirred by magnetic stirrer under nitrogen atmosphere. The polymerization time was two hours. The sample was cooled and dialysed against water for a week and then was freeze-dried in a Virtis Freeze Drier (CHRIST ALPHA 1-2) under vacuum at −52° C. for 4 days.

Synthesis of Hydrogels and Nanocomposites (photopolymerization)

HEMA-PEGDMA hydrogels were prepared in aqueous solution. The total concentration of the monomers was 30 wt %. Hydrogels were prepared using different feed mol ratios as 90/10, 75/25, 50/50, 25/75 and 10/90. In addition 1.0 mol % of photoinitiator calculated for monomers was measured and these solutions were homogenized by hand and then by ultrasonic bath. The NCHGs were made by the same method in which the HEMA/PEGDMA feed was 50/50 and 15 wt % NPs was dispersed in the clear, yellowish aqueous solution of monomers and photoinitiator. The prepared NPs were loaded with CHX, and allowed to swell for 48 hours. Then these loaded particles were freeze-dried. The solid, loaded particles were then dissolved in the aqueous solution of monomers and initiator in order to form the NCHGs.

The mixtures were poured into cylindrical molds made of polypropylene, with a diameter of 9 mm and the prepared sample height varied from 4 mm to 10 mm. The tops of the holders were closed with cover slips to avoid the inhibition effect of the oxygen. The initiation of photopolymerization was performed by Kulzer Palatray CU lamp source supplying blue light with 420 nm wavelength and 1.50 watt per cm² irradiation energy. The reaction time was adjusted for 25 min due to the larger thickness of the specimen. CHX was added to the reaction mixture prior to photopolymerization. In the case of matrix gels without NPs, CHX was dissolved in the aqueous solution of monomers and then crosslinked. For NCHGs, the NPs were swelled in aqueous solution of CHX and then added to the mixture of monomers and initiator, and then crosslinked. The composition of gel particles is summarized in Table 1.

Mechanical Assay

Hydrogel specimens were prepared according to the method described above. Samples were investigated with INSTRON 4302 Mechanical Analyser. The compression tests were performed on cylindrical samples described above with the full scale load range at 0.1 kN, and the crosshead speed at 2 mm/min. The cylindrical samples had a diameter of 9 mm and specimen length of 4 and 9 mm, for the matrix hydrogel and for NCHG systems, respectively.

Dynamic Light Scattering (DLS).

Hydrodynamic diameter (H_{D}) of NPs was measured with a BI-200SM Brookhaven Research Laser Light Scattering photometer equipped with a NdYAg solid state laser at an operating wavelength of □₀₌532 nm. Measurements of the average size of NPs were performed at 25° C. with an angle detection of 90° in optically homogeneous quartz cylinder cuvettes. The samples were prepared from the reaction mixture after dialysis, and from freeze-dried samples. The concentration of the polymer solutions was 100 □g/ml.

Scanning Electron Microscopy (SEM) Analysis

The hydrogels were dried at 110° C. for 2 hours and sputter-coated with gold twice for approximately 30 sec to a thickness of the coating was approximately 100 nm, the plasma current was 18-20 mA, and the pressure was 10⁻² mPa. Samples were imaged using scanning electron microscope (Hitachi S4300 CFE, Tokyo, Japan, with W emitter) at 1.5 and 10 kV.

Swelling Measurements

The swelling experiments were carried out by immersion of hydrogels specimen (9.0 mm diameter and 4.0 mm height) in distilled water. At definite intervals of time, the samples were removed from water and wiped with bolting paper to eliminate the excess water. The measurements were iterated until the hydrated gels achieved a constant weight value. The weight swelling percentage (Wₚ) for each sample was calculated as: Wₚ=(Wₛ−W_{d})/W_{d}×100; where Wₛ is the weight of the swollen gel and the W_{d} is the original weight of the gel after polymerization.

Release Studies

The matrix hydrogels and NCHGs containing CHX (cylindrical geometry 9 mm×4 mm) were prepared for release studies with the composition summarized in Table 1. The main purpose of these experiments was to analyse the release rate of the drug from the loaded matrices, from NCHG1 where CHX was only in the matrix, only in the NPs (NCHG2), and finally in both components (NCHG3). For this purpose the NCHG matrices were loaded with 15 mg of CHX, the NPs were loaded with 45 mg of CHX. The NCHGs were loaded with a total of 60 mg of the model drug. The investigated samples were immersed in distilled water (35 ml) and subjected to continuous magnetic stirring. At regular time intervals, an aliquot of 0.5 ml was removed, and the concentration of CHX was measured by HPLC.

Measurements of Drug Concentration by HPLC

The concentration of the released CHX was determined by HPLC on a Merck-Hitachi LaChrom instrument using C18 column, and UV detection at 257 nm. The mobile phase was 35% acetonitrile and 65% 20 mM acetate buffer with pH=3.8, and the flow rate was 0.5 ml/min.

Results

CHX has been used routinely to treat periodontal disease. However, when applied locally it quickly disperses. By incorporating CHX into a polymer matrix, from which it can be slowly released, it can be more efficacious.

Preparation of Nanoparticles

NPs were formed from a composition of HEMA/PEGDMA=50/50 mol %. The crosslinking density of the NPs is variable, which may effect the rate of release. Here the vinyl groups of HEMA were crosslinked with the divinyl monomer of PEGDMA by free radical polymerization in micellar polymerization, forming stable NPs.

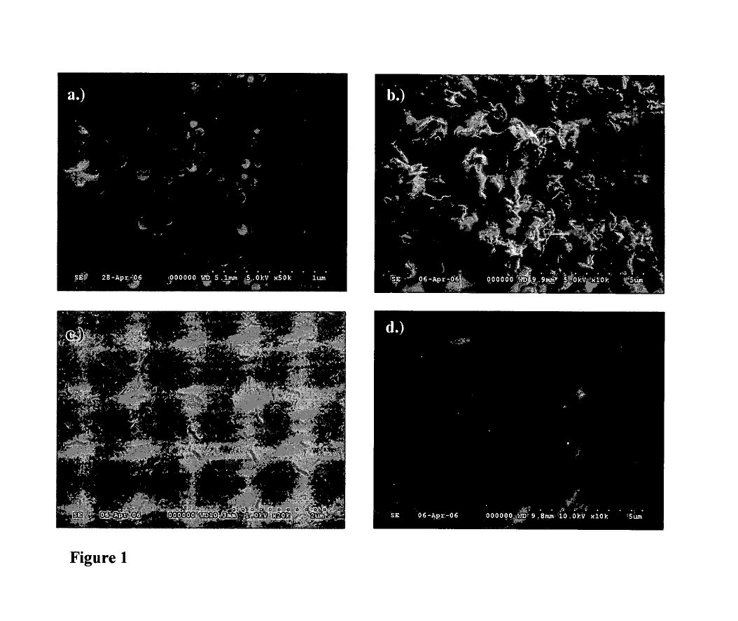

The particle size of HEMA-PEGDMA NPs was determined by SEM and DLS measurements. SEM micrographs of crosslinked NPs of copolymer were taken from the colloid solution and freeze-dried form, using a concentration of 50 μgm/ml. SEM micrographs (FIG. 1a) confirmed spherical, nanosized copolymer particles. The size of dried particles was in the range of 50-150 nm. The DLS measurements demonstrated that the NPs have a size distribution from 5 nm to 500 nm.

Synthesis of Hydrogel and Nanocomposite (photopolymerization)

CHX loaded NCHGs which can release the drug in a slow manner compared to the matrix hydrogels were prepared. Because the inner structure, porosity of the gel is the most important parameter for the release properties, present hydrogels were investigated with different composition. The total organic phase was 30 wt % in the aqueous solutions. The ratio of HEMA was varied from 90 mol % to 10 mol % while the ratio of PEGDMA crosslinker was varied from 10 mol % to 90 mol %. The polymer solution weighed 2 g and five gels were prepared for parallel release measurements. The produced materials are yellow or white yellow, soft and flexible hydrogels with cylindrical shape. The transparency was increased with increasing amount of PEGDMA.

In the NCHG the amount of monomers in the hydrogel was constant although it could be varied. The ratio of NPs was 15 wt % vs. the total mass of the hydrogel, and 50 wt % vs. amount of monomers (Table 1). The composite gels were more consistent than matrix gels and were more rigid and more transparent.

Mechanical Assay

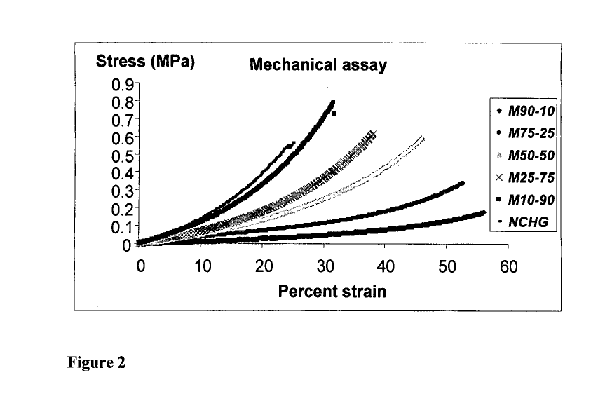

The assay of mechanical properties was repeated ten times to ensure reliable results. The matrix hydrogels were investigated in five different mol ratios (Table 2). When the amount of crosslinker was increased, the compression strength also increased and, depending on the cross-linker density, by as much as in excess of 400% comparing sample 1 to sample 5. In the case of matrix gels the compressive strength of sample 1 (HEMA/PEGDMA 9/1) changed from 0.18 MPa to 0.59 MPa for sample 3 (HEMA/PEGDMA 5/5) and than to 0.79 MPa for sample 5 (HEMA/PEGDMA 9/1). The compression strength values are very similar for the NCHG was 0.56 MPa compared to the 0.59 MPa value of the 50/50 matrix gel (sample 3). Nevertheless the strain was altered accordingly, because when the ratio of PEGDMA was only 10% and 25% the samples were very soft, but when it was augmented (50, 75 or 90%) the specimens were brittle. The gels were harder and more rigid when the amount of crosslinker was increased. For the NCHG it was observed that the compression strength value does not change related to the matrix, however the flexibility decreases. The shape of compressive strengths values is shown the FIG. 2.

SEM Analysis

SEM micrographs of hydrogels, NPs and NCHGs are shown in FIG. 1, a-d. The picture of the hydrogel (FIG. 1b) shows evidence that two types of surfaces are observed on the gel. These types do not differentiate from each other, and form the entire matrix. Either a more angular part is pronounced as angled shapes, or it is embed in another rounded environment. This dual disposition is generally representative for the entire matrix.

SEM image of the surface of the NCHG is shown in FIG. 1c. The picture taken with 20000 magnification shows a relatively smooth surface where only divots can be observed. The size of these cracks are from 100 nm to 200 nm but could not discovered other formations. The broken sample is analysed in the FIG. 1d, this picture shows large number of NPs in the matrix. This image shows particles with a size of about 200 nm ball-shaped nanoparticles inside the matrix.

Swelling Ratios

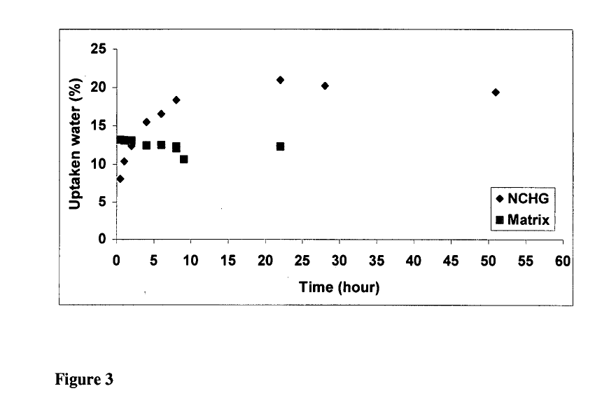

This swelling procedure was studied for five parallel samples in case of hydrogel and in three for the NCHG. The representative swelling kinetic curves for hydrogels and NCHGs are shown in FIG. 3. The curves show that the weights of hydrogels after photopolymerization do not increase notably, it is about 13% in the first half hour and later an equilibrium state was observed. In contrast, the swelling behaviour of NCHG is different. It swelled slower in the first two hours, but after this the swelling was continued till 22 hours when the samples have reached equilibrium weight. It seems that the final swelling capacity of the NCHG is higher (about 21%) but the water uptake is slower comparing to the matrix hydrogel. This remarkable swelling rate would be a favourable property, these kind of materials can be usable for dental applications.

Release Properties

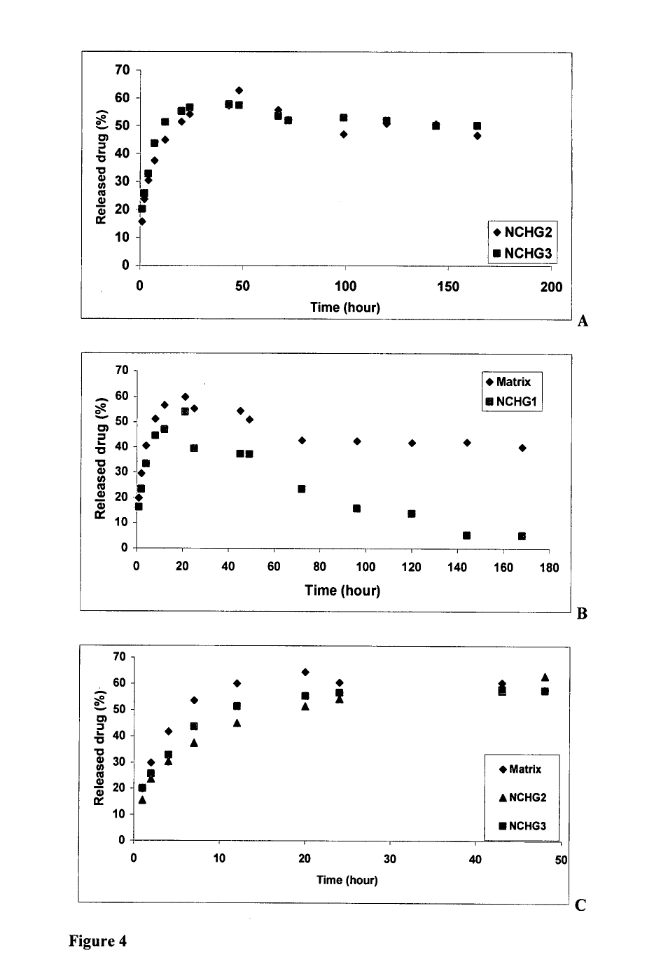

The release curves of CHX from basic hydrogel (50% HEMA and 50% PEGDMA) and from the NCHG are shown in FIG. 4. The release profiles were investigated in the case of matrix and for the NCHG with variation of the reservoir. Samples were loaded as follows: only the matrix was (NCHG1), only into the NPs (NCHG2), and when both of these components contained drug (NCHG3). The amount of NPs was 15 wt % in all samples and the loaded drug was 45 mg in the NPs, and 15 mg in the matrix. The measurements were performed for three parallel experiments to ensure reliable results. In the first four hours the difference was not too considerable, but after the seventh hour remarkable difference was observed. The release from the matrix gel was the fastest than the NCHG where only the matrix was loaded (FIG. 4a). Accordingly, the effect of NP could be followed in the initial period. This controlled release was continued to forty-eight hours after the degree of delivered drug closes to consistent (FIG. 4b, and extended part in FIG. 4c). The maximum ratio of the released drug in each case reached up to 60% of loaded drug, the main difference is altogether the time. For the NCHG1 sample with loaded matrix and empty NPs shows an unexpected profile. In the first period CHX is released, but then its concentration declines. The NPs entrapped a part of the drug. The reason of this phenomenon has not been understood yet.

TABLE 1
Composition of nanocomposites and matrix gels prepared
for release analysis. In the matrix gel (5/5: HEMA/PEGDMA)
CHX was 15 mg; in NCHG1 the CHX was 15 mg only in the
matrix; in NCHG2 CHX was 45 mg only in NPs; in NCHG3 the
total amount of CHX was 60 mg divided in two parts 15
mg in the matrix and 45 mg in NPs was loaded.

| Sample | HEMA | PEGDMA | Initiator | NPs | CHX 20% | H₂O |
|---|---|---|---|---|---|---|
| number | (g) | (g) | (g) | (g) | (ml) | (g) |
| Matrix gel | 0.1069 | 0.4930 | 0.0051 | 0 | 0.075 | 1.32 |
| NCHG1 | 0.1069 | 0.4930 | 0.0051 | 0.3 | 0.075 | 1.02 |
| NCHG2 | 0.1069 | 0.4930 | 0.0051 | 0.3 | 0.225 | 0.87 |
| NCHG3 | 0.1069 | 0.4930 | 0.0051 | 0.3 | 0.075 + | 0.795 |
| | | | | | 0.225 | |

TABLE 2
The summary of results of mechanical assays
of matrix hydrogel samples (1-5) and NCHG.

| | Number of sample | | | | | |
|---|---|---|---|---|---|---|
| | 1 | 2 | 3 | 4 | 5 | NCHG |
| Ratio of HEMA- | 90-10 | 75-25 | 50-50 | 25-75 | 10-90 | 50-50 + |
| PEGDMA | | | | | | 30% NPs |
| Percent strain (%) | 56.5 | 53.1 | 46.4 | 38.5 | 32.0 | 25.3 |
| Stress at maximum | 0.18 | 0.34 | 0.59 | 0.61 | 0.79 | 0.56 |
| (MPa) | | | | | | |

What is claimed is:

1. A nanocomposite biocompatible hydrogel comprising a gel matrix containing nanoparticles interspersed throughout the matrix and an antibacterial composition in said nanoparticles.

2. The hydrogel according to claim 1 wherein said antibacterial material is chlorhexidine-di-gluconate.

3. The hydrogel according to claim 2 wherein said nanoparticles are the product of the reaction of 2-hydroxyethyl methacrylate (HEMA) and polyethyleneglycol demethacrytate (PEGDMA).

4. The hydrogel according to claim 3 wherein the nanoparticles are formed by means of free radical initiated copolymerization of HEMA and PEGDMA in an aqueous solution.

5. The hydrogel according to claim 3 wherein said nanoparticles are formed by photopolymerization of HEMA and PEDGMA.

6. The hydrogel according to claim 3 wherein the HEMA is present in the nanoparticle in the range of about 1% to 99% by weight and the PEGDMA is present in the nanoparticle in the range of about 99% by weight to 1% by weight.

7. The hydrogel according to claim 6 wherein the gel matrix is formed by photopolymerization of HEMA and PEGDMA.

8. The hydrogel according to claim 1 wherein the gel matrix, nanoparticles and antibacterial material are blended together and blend is cross linked.

9. The hydrogel according to claim 7 wherein the gel matrix, nanoparticles and chlorhexidine are blended together and the blend is cross linked.

10. The hydrogel according to claim 9 wherein the cross linking is by photo-polymerization.

11. A nanocomposite biocompatible hydrogel comprising a HEMA-PEGDMA hydrogel matrix prepared in an aqueous solution of about 1 to 99 HEMA and about 99% to about 1% PEGDMA, said matrix further comprising nanoparticles formed from the copolymerization reaction HEMA and PEGDMA in an aqueous solution and chlorhexidine-di-gluconate.

12. A method of making nanocomposite biocompatible hydrogels comprising
forming nanoparticles comprised of the product of a copolymerization reaction of HEMA and PEGDMA
loading said nanoparticles with chlorhexidine-di-gluconate
blending said nanoparticles with HEMA and PEGDMA in an aqueous solution polymerizing the blend to form a gel matrix.

13. The method according to claim 12 wherein said nanoparticles are formed by a copolymerization reaction in an aqueous solution.

14. The method according to claim 13 wherein the nanoparticles loaded with chlorhexidine-di-gluconate are dissolved in a solution containing a photoinitiator.

15. The method according to claim 14 wherein said blend is polymerized by photopolymerization.

16. A drug delivery system comprising a HEMA-PEGDMA hydrogel matrix prepared in an aqueous solution of about 1 to 99 HEMA and about 99% to about 1% PEGDMA, said matrix further comprising nanoparticles formed from the copolymerization reaction HEMA and PEGDMA in an aqueous solution and chlorhexidine-di-gluconate.

17. A softgel for implantation into a periodontal pocket comprising a HEMA-PEGDMA hydrogel matrix prepared in an aqueous solution of about 1 to 99 HEMA and about 99% to about 1% PEGDMA, said matrix further comprising nanoparticles formed from the copolymerization reaction HEMA and PEGDMA in an aqueous solution and chlorhexidine-di-gluconate.

18. A surface film for the treatment of infected gums, said film comprising a HEMA-PEGDMA hydrogel matrix prepared in an aqueous solution of about 1 to 99 HEMA and about 99% to about 1% PEGDMA, said matrix further comprising nanoparticles formed from the copolymerization reaction HEMA and PEGDMA in an aqueous solution and chlorhexidine-di-gluconate.

19. A method according to claim 12 wherein said polymerization of the blend is performed in situ in a patient.

20. A method according to claim 19 wherein said polymerization is photopolymerization.
